(19) United States
(12) Patent Application Publication
(10) Pub. No.: US 2008/0113350 A1
(43) Pub. Date: May 15, 2008

(54) Blood test to monitor the genetic changes of progressive cancer using immunomagnetic enrichment and fluorescence in situ hybridization (FISH)

(76) Inventor: Leon W.M.M. Terstappen, Huntingdon Valley, PA (US)

Correspondence Address: IMMUNICON CORPORATION, 3401 MASONS MILL ROAD, SUITE 100, HUNTINGDON VALLEY, PA, 19006, US

(21) Appl. No.: 11/595,504

(22) Filed: Nov. 9, 2006

(51) Int. Cl. C12Q 1/68

(52) U.S. Cl. 435/6

(57) ABSTRACT

Amplification and overexpression of theHER-2 oncogene in breast cancer is felt to be stable over the course of disease and concordant between the primary tumor and metastases. Therefore, patients with HER-2 negative primary tumors will rarely receive anti-HER-2 antibody therapy. A very sensitive blood test is used to capture circulating tumor cells (CTC's) and evaluate their HER-2 gene status by FISH evaluation. The HER-2 status of the primary tumor and corresponding CTC's is used to assess the ratio of CTC's as a reliable surrogate marker. HER-2 expression of 10 CTC's is sufficient to make a definitive diagnosis of the HER-2 gene status for the whole population of CTC's in patients with recurrent breast cancer.

### CROSS-REFERENCE TO RELATED APPLICATIONS

This is a non-provisional application which claims priority to U.S. 60/573,801, filed on May 24, 2004

### BACKGROUND

1. Field of the Invention

The invention relates generally to cancer prognosis and survival in metastatic cancer patients, based on the presence of morphologically intact circulating cancer cells (CTC) in blood. More specifically, diagnostic methods, reagents and apparatus are described that correlate the overexpression and/or amplification of HER-2 in a blood sample with CTC as an accurate and predictive indicator of therapeutic response.

2. Background Art

Despite efforts to improve treatment and management of cancer patients, survival in cancer patients has not improved over the past two decades for many cancer types. Accordingly, most cancer patients are not killed by their primary tumor, but they succumb instead to metastases: multiple widespread tumor colonies established by malignant cells that detach themselves from the original tumor and travel through the body, often to distant sites. The most successful therapeutic strategy in cancer is early detection and surgical removal of the tumor while still organ confined. Many cancers detected at early stages have established micrometastases prior to surgical resection. Thus, early and accurate determination of the cancer's malignant potential is important for selection of proper therapy. Properly developed diagnostic and prognostic data banks in the treatment and detection of metastatic cancer focusing on survival provides an enormous benefit to medicine (U.S. Pat. No. 6,063,586).

### Detection HER-2/new as a Soluble Tumor Antigen:

Increased HER2/neu results in decreased response to hormone therapy, and is a significant prognostic factor in predicting responses to hormone receptor-positive metastatic breast cancer. Thus in malignancies where the HER2/neu oncogene product is associated, methods have been described to monitor therapy or assess risks based on elevated levels (U.S. Pat. No. 5,876,712). Unfortunately, the base levels during remission or in healthy normals are relatively high and may overlap with concentrations found in patients, thus requiring multiple testing and monitoring to establish patient-dependent baseline and cut-off levels. Thus, there is a need for a reliable blood test to characterize HER-2 in blood.

### HER-2 as a Genetic Marker:

There is considerable clinical data demonstrating that HER2/neu overexpression, usually attributable to HER-2 gene amplification, occurs in approximately 20-25% of breast cancer patients and is associated with a poor prognosis. The diagnosis of HER-2 overexpression and/or HER-2 gene amplification is made on the primary tumor, and provides the “gold” standard for assessing the HER-2 status in primary tumors. Comparison of the immunohistochemical methods determine overexpression and fluorescence in situ hybridization (FISH) to determine gene amplification in these tumors has shown that the latter is more accurate and more predictive of a favorable response. The 70-75% of patients who do not have HER-2 gene amplification in their primary tumors are rarely diagnosed with such amplification at a later date because biopsies are done infrequently and usually not examined for HER-2 status. Therefore, if HER-2 gene amplification is detected, it is important to develop a safe and definitive method for making this diagnosis so that such patients can receive optimal treatment.

### Assessment of Intact Tumor Cells in Cancer Detection and Prognosis:

Immunomagnetic separation technology provides greater sensitivity and specificity in the unequivocal detection of intact circulating cancer cells. This simple and sensitive diagnostic tool, as described (U.S. Pat. No. 6,365,362; U.S. Pat. No. 6,551,843; U.S. Pat. No. 6,623,982; U.S. Pat. No. 6,620,627; U.S. Pat. No. 6,645,731; PCT/US02/06967; PCT/US02/05233; PCT/US02/26861; PCT/US04/005848; and PCT/US05/08602) is used in the present invention as enrichment means for CTC's in a blood sample. The quantitation of CTC in a blood sample has been shown to have prognostic potential in assessing overall/progression-free survival and response to therapy in metastatic cancer patients.

Using this diagnostic tool, a blood sample from a cancer patient is incubated with magnetic beads, coated with antibodies directed against an epithelial cell surface antigen as for example EpCAM. After labeling with anti-EpCAM-coated magnetic nanoparticles, the magnetically labeled cells are then isolated using a magnetic separator. The immunomagnetically enriched fraction can be further processed for downstream immunocytochemical analysis or image cytometry, for example, in the Cell Spotter® System (Immunicon Corp., PA). The magnetic fraction can also be used for downstream immunocytochemical analysis, RT-PCR, PCR, FISH, flowcytometry, or other types of image cytometry. In the present invention, HER-2 analysis by FISH provides an analytical tool to asses HER-2 expression and potential chemotherapeutic response.

One embodiment of the present invention includes a sensitive blood test to detect and characterize CTC's after immunomagnetic selection and separation to highly enrich and concentrate any epithelial cells present in whole blood samples, coupled with HER-2 FISH as more accurate and predictive of current immunohistochemical methods. The captured cells are detectably labeled with a leukocyte specific marker and with one or more tumor cell specific fluorescent monoclonal antibodies to allow identification and enumeration of the captured CTC's as well as unequivocal differentiation from contaminating non-target cells with the detection of HER-2. The embodiment of the present invention is not limited to image cytometry, but includes any isolation and imaging protocol, coupled to HER-2 FISH analysis.

HER-2 amplification and overexpression are associated with poor prognosis in patients with breast cancer, and is therefore an important therapeutic target. A tumor's HER-2 status is generally considered to be stable, but expression levels are known to change throughout disease progression. This means that patients who do not initially respond to anti-HER-2 therapy might do so at a later stage.

HER-2 is amplified and overexpressed in up to 25% of breast tumors. The humanized monoclonal antibody trastuzumab can effectively treat these patients when it is administered either as a monotherapy or in combination with chemotherapy. HER-2 overexpression, however, is usually determined based on examination of the primary tumor. Patients whose tumors are not initially found to overexpress HER-2 are no longer considered for trastuzumab therapy, and repeated biopsies are not usually performed to evaluate the additional changes that accompany cancer progression.

With a need to consistently and reliably assess the HER-2 status in patients and with the ability to acquire Her-2 gene amplification, the present invention provides a safe and definitive method for making this diagnosis so that such patients can receive optimal treatment.

### SUMMARY OF THE INVENTION

The present invention is a method and means to detect and characterize circulating tumor cells (CTCs). CTCs are detected in a majority of primary tumors and in patients with a recurrence of breast cancer either during treatment, between therapeutic regimens or when patients are chemorefractory and the tumor is progressing. A major obstacle in treating any tumor is that the tumor cells are constantly changing, leaving the oncologist to base a therapeutic regimen on a biopsy. The small percentage of biopsies that are performed are infrequently investigated for HER-2 overexpression and repeated biopsies cannot be performed to evaluate the additional changes that are likely to accompany cancer progression. Also, metastases which can be mono- or polyclonal can differ from one another with regard to HER-2 status. In contrast, obtaining a blood sample is safe and can be performed repeatedly. Analysis can be automated, and yield more valid HER-2 gene ratios to aid in diagnosis. The present invention quantitates signals from FISH examination of CTCs which are non-overlaping and flattened against the slide. The result is that Her-2 gene amplification is accurately measured in individual cells. This method is used to determine concordance between the pathologist's analysis of Her-2 gene status in primary tumors and corresponding CTCs. Thus, assessing Her-2 gene amplification in isolated CTC's with tumor progression provides a tool to assess such patient's ability to respond to targeted therapy.

### BRIEF DESCRIPTION OF THE DRAWINGS

FIG. 1: Criteria for identification of a CTC include cytomorphology, immunophenotype and aneusomy. Panel A shows a CTC. Panel B shows anti-CK stained CTC probed by FISH.

### DETAILED DESCRIPTION OF THE INVENTION

The object of this invention is to provide a means for assessing HER-2 expression in patients with metastatic breast cancer, and using this information as a tool for determining individual patient response to therapy.

Under the broadest aspect of the invention, there is no limitation on the collection and handling of samples as long as consistency is maintained. Accordingly, the cells can be obtained by methods known in the art.

While any effective mechanism for isolating, enriching, and analyzing CTCs in blood is appropriate, one method for collecting circulating tumor cells combines immunomagnetic enrichment technology, immunofluorescent labeling technology with an appropriate analytical platform after initial blood draw. The associated test has the sensitivity and specificity to detect these rare cells in a sample of whole blood and to investigate their role in the clinical course of the disease in malignant tumors of epithelial origin. Further assessment using HER-2 FISH analysis provides a very sensitive blood test for evaluating HER-2 gene status. From a sample of whole blood, rare cells are detected with a sensitivity and specificity to allow them to be collected and used in the diagnostic assay of the invention, namely assessing HER-2 expression status in CTC's of patients diagnosed with metastatic breast cancer. The present invention provides a means for assessing the response to therapy of the disease through the concordance between the primary tumor and metastases.

Circulating tumor cells (CTC) have been shown to exist in the blood in detectable amounts. This created a tool to investigate the significance of cells of epithelial origin in the peripheral circulation of cancer patients (Racila E., Euhus D., Weiss A. J., Rao C., McConnell J., Terstappen L. W. M. M. and Uhr J. W., Detection and characterization of carcinoma cells in the blood, Proc. Natl. Acad. Sci. USA, 95:4589-4594 (1998)). This study demonstrated that these blood-borne cells might have a significant role in the pathophysiology of cancer. Having a detection sensitivity of 1 epithelial cell per 5 ml of blood, the assay incorporates immunomagnetic sample enrichment and fluorescent monoclonal antibody staining followed by flowcytometry for a rapid and sensitive analysis of a sample. The results show that the number of epithelial cells in peripheral blood of eight patients treated for metastatic carcinoma of the breast correlate with disease progression and response to therapy. In 13 of 14 patients with localized disease, 5 of 5 patients with lymph node involvement and 11 of 11 patients with distant metastasis, epithelial cells were found in peripheral blood. The number of epithelial cells was significantly larger in patients with extensive disease.

The assay was further configured to an image cytometric analysis such that the immunomagnetically enriched sample is analyzed by image cytometry (see Example 1). This is a fluorescence-based microscope image analysis system, which in contrast with flowcytometric analysis permits the visualization of events and the assessment of morphologic features to further identify objects.

Automated fluorescence microscopic system, used for automated enumeration of isolated cells from blood, allows for an integrated computer controlled fluorescence microscope and automated stage with a magnetic yoke assembly that will hold a disposable sample cartridge. The magnetic yoke is designed to enable ferrofluid-labeled candidate tumor cells within the sample chamber to be magnetically localized to the upper viewing surface of the sample cartridge for microscopic viewing. Software presents suspect cancer cells, labeled with antibodies to cytokeratin and having epithelial origin, to the operator for final selection.

While isolation of tumor cells can be accomplished by any means known in the art, cells can be stabilized, prior to enrichment. Epithelial cell-specific magnetic particles are added and incubated for 20 minutes. After magnetic separation, the cells bound to the immunomagnetic-linked antibodies are magnetically held at the wall of the tube. Unbound sample is then aspirated and an isotonic solution is added to resuspend the sample. A nucleic acid dye, monoclonal antibodies to cytokeratin (a marker of epithelial cells) and CD 45 (a broad-spectrum leukocyte marker) are incubated with the sample. After magnetic separation, the unbound fraction is again aspirated and the bound and labeled cells are resuspended in an isotonic solution. The sample is suspended in a cell presentation chamber and placed in a magnetic device whose field orients the magnetically labeled cells for fluorescence microscopic examination. Cells are identified automatically and candidate circulating tumor cells presented to the operator for checklist enumeration. An enumeration checklist consists of predetermined morphologic criteria constituting a complete cell (see example 1).

The diagnostic potential of immunomagnetic enrichment and image cytometry, together with the use of intact circulating tumor cells as a prognostic factor in cancer survival, can provide a rapid and sensitive method for determining appropriate treatment. When this is coupled with HER-2 FISH of individual isolated CTC's on prepared cover slips, a very sensitive means for assessing the presence of HER-2 expression is metastatic breast cancer patients is presented as a diagnostic surrogate.

The following examples illustrate the predictive and prognostic value of CTC's in blood from patients, and the potential diagnostic significance of CTC's expressing the HER-2 gene. Note, the following examples are offered by way of illustration and are not in any way intended to limit the scope of the invention.

### EXAMPLE 1

### Enumeration of Circulating Cytokeratin Positive Cells Using CellPrep™

Cytokeratin positive cells are isolated by a cell preservative system using a 7.5 ml sample of whole blood. Epithelial cell-specific immunomagnetic fluid is added and incubated for 20 minutes. After magnetic separation for 20 minutes, the cells bound to the immunomagnetic-linked antibodies are magnetically held at the wall of the tube. Unbound sample is then aspirated and an isotonic solution is added to resuspend the sample. A nucleic acid dye, monoclonal antibodies to cytokeratin (a marker of epithelial cells) and CD 45 (a broad-spectrum leukocyte marker) are incubated with the sample for 15 minutes. After magnetic separation, the unbound fraction is again aspirated and the bound and labeled cells are resuspended in 0.2 ml of an isotonic solution. The sample is suspended in a cell presentation chamber and placed in a magnetic device whose field orients the magnetically labeled cells for fluorescence microscopic examination. Cells are identified automatically; control cells are enumerated by the system, whereas the candidate circulating tumor cells are presented to the operator for enumeration using a checklist.

### EXAMPLE 2

### HER-2 Gene Amplification Acquisition with Breast Cancer Progression

CTC's from patients with metastatic breast cancer are enriched and isolated as described in Example 1. After blood samples are treated, 2 mm EDTA is added to the buffer. The cells were not permeabilized. The samples are washed, the supernatant aspirated and resuspended in 100 μl/5 ml of blood of phosphate buffered saline. 100 ul is placed on a slide and air-dried at 37° C. Slides are stored at −80° C.

Multicolor FISH (MCF) is performed by pretreatment and denaturation of slides prior to incubation with HER-2 specific probes. Hybridization and post-hybridization washes are performed by standard procedures in the art. Slides are counterstained and prepared with mounting media containing DAPI.

Concordance between HER-2 status, tumor and CTC's are analyzed by binomial distribution.

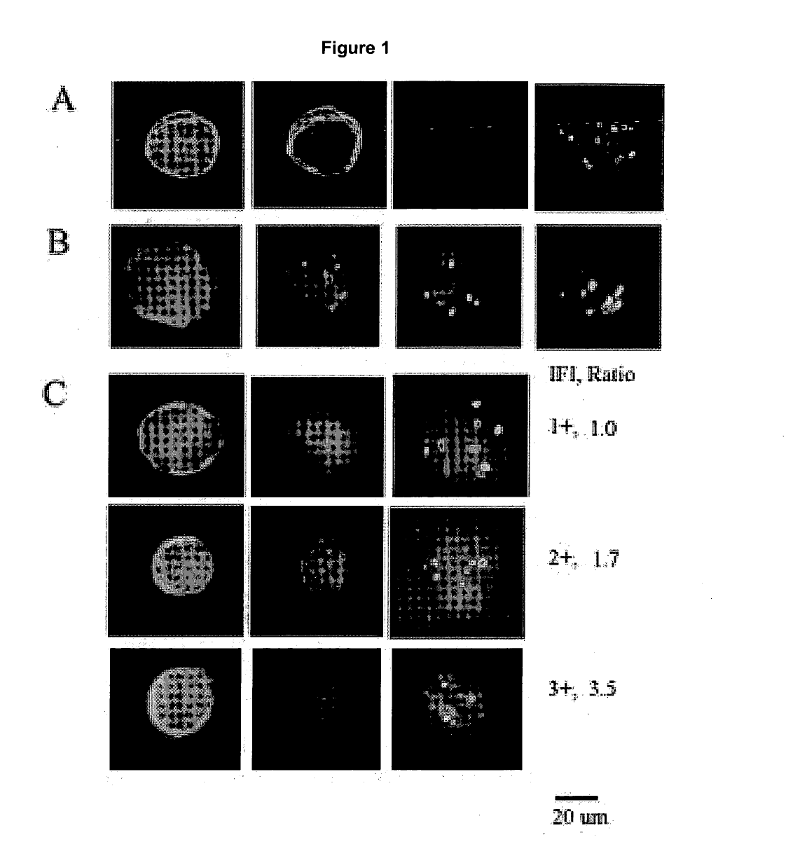

Identification of a CTC includes cytomorphology, immunophenotype and aneusomy. FIG. 1A shows a classical CTC: large round cell, high nuclear to cytoplasmic ratio, staining of the periphery of cells with anti-CK, anti-mammaglobin staining (anti-HER-2 is also considered) of both periphery and cytoplasm of cells, and no staining with anti-CD45, a WBC marker. FIG. 1B shows anti-CK staining and aneusomy in a CTC probed 3 times by FISH. In the present example, anti-HER2 was used, along with CEP17 and HER-2 are the DNA probes.

There is a concordance between the blood test and that obtained from the primary tumor. 97% concordance was obtained. After dividing the isolated CTC's into bins containing 10 consecutive CTC's where each bin in a patient would reflect the gene status of the patient, the results show that 139 of 141 bins are concordant with the overall HER-2 gene status. Other bin configurations are considered in the present invention.

The number of CTC's sufficient for correctly calculating the HER-2 status and diagnosis are determined for a large number of patients using CTC thresholds from ROC curves.

In patients whose primary tumor was HER-2 negative and developed recurrence, HER-2 gene amplification in CTC's have ratios between 2.0 and 2.7. When these ratios are compared with each HER-2 positive primary tumor and its corresponding CTC's, HER-2 gene ratios in the primary tumor is 2.44 fold higher than the corresponding CTC's, indicating that the comparatively low ratio of HER-2 in CTC's are a consistently reliable surrogate marker for the higher gene amplification of the corresponding tumor.

Clinical evaluation is assessed with clinical responses in conjunction with biochemical responses, monitored with surrogate markers for response or progression. The CTC's that were HER-2 amplified were preferentially eliminated in patients treated with Herceptin as well as Cisplatin.

Evaluation HER-2 status in the present invention begins with immunohistochemistry (IHC) for the expression of HER-2 protein. Using a high affinity murine anti-human HER-2 protein (HER-81) along with a nucleic acid dye (DAPI), anticytokeratin-FITC and anti-CD45, HER-2 protein is stained on the CTC's from patients with metastatic breast cancer. Three different densities of HER-2 protein were distinguishable (FIG. 1C). CTC's are then placed in subsets of 10 consecutive bins, and the average HER-2 expression and amplification calculated for each bin. Concordance of all bins was obtained within each patient. Consequently HER-2 expression predicts HER-2 gene amplification with high probability.

The advantage of CTC analysis of HER-2 expression over biopsy of tumors are that the individual cells can be ascertained with the acquisition of a blood sample. In this way, examination of individual CTC's could quantify the number of CTC's in a subset that is amplified for each gene. The results indicate that a combination of targeted drugs should affect the CTC's and, therefore, the appropriate targeted drugs should be given in combination. Further, examination of individual cells allows comparison of immunofluoresence intensity of anti-HER2 flouorochrome with the precise HER2/CEP17 ratio. The correlation between intensity of staining and gene amplification is readily studied. With a need to monitor HER-2 expression in patient groups where biopsies of the primary tumor are HER-2 negative and subsequent rise in the number of CTC's with very high levels of HER2/CTC ratios, there is the possibility that monitoring HER-2 gene amplification in CTC during progression of the cancer provides a window for clinical assessment. The present invention provides a means for oncologists to examine patient status in treatment. Further, automated analysis yields moren valid HER-2 gene ratios which aid in the pathological diagnosis.

What is claimed is:

1. A method for assessing individual patient response to anti-HER-2 therapy comprising:
a) obtaining a biological specimen from diagnosed with metastatic breast cancer, said specimen comprising a mixed cell population suspected of containing rare cells;
b) immunomagnetically enriching a fraction of said specimen, said fraction containing said rare cells;
c) confirming at least HER-2 protein expression in said rare cells by immunofluorescence;
d) quantitating at least HER-2 gene expression in said rare cells by FISH; and
e) correlating said gene expression and said protein expression with rare cell count to predict therapeutic response based upon a predetermined statistical association.

2. A method as claimed in claim 1, wherein said fraction is obtained by immunomagnetic enrichment of colloidal paramagnetic particles, coupled to a biospecific ligand which specifically binds to said rare cells, to the substantial exclusion of other populations.

3. A method as claimed in claim 2, wherein said rare cells are immunomagnetically enriched with antibodies to the epithelial cell surface antigen EPCAM.

4. A method as claimed in claim 1, wherein HER-2 and CEP17 are DNA probes used in said FISH.

5. A method as claimed in claim 1, wherein said correlating is a statistical comparison of the immunofluoresent intensity of said anti-HER-2 protein expression and said FISH in said rare cells.

6. A method as claimed in claim 1 whereby said correlating provides phenotypic and genotypic information of said rare cells for assessing therapy in patients.
